(19) United States
(12) Patent Application Publication
(10) Pub. No.: US 2009/0024129 A1
(43) Pub. Date: Jan. 22, 2009

(54) PERFORATOR WITH INNER AND OUTER DRILLS AND A DRIVE HEAD, THE INNER DRILL CONFIGURED TO MOVE AGAINST THE OUTER DRILL IN ORDER TO DISENGAGE FROM THE DRIVE HEAD

(76) Inventors: Jeremy Gordon, Madison, WI (US); Kevin Manley, Cobh (IE); Heather Gordon, Paw Paw, MI (US)

Correspondence Address: INTEL. PROP./ RND;STRYKER CORPORATION, 4100 EAST MILHAM AVE., KALMAZOO, MI, 49001-6197, US

(21) Appl. No.: 11/780,172

(22) Filed: Jul. 19, 2007

(51) Int. Cl. A61B 17/16

(52) U.S. Cl. 606/80; 408/225; 606/173; 606/180

(57) ABSTRACT

A cranial perforator with an inner drill and an outer drill that surrounds the inner drill. Both drills are rotated by a head. Once the inner drill penetrates the bone, the inner drill moves against the outer drill. This movement causes the inner drill to disengage from the perforator head. The disengagement of the inner drill from the perforator head serves to stop the rotation of both drills.

### FIELD OF THE INVENTION

This invention is generally related to a medical perforator such as a cranial perforator. More particularly, this invention is related to a medical perforator with inner and outer drills, wherein the inner drill moves against the outer drill to cause both drills to disengage from the drive head.

### BACKGROUND OF THE INVENTION

A perforator is a medical device designed to cut through tissue. One such perforator is a cranial perforator. In a neurological surgical procedure, the cranial perforator is used to form the initial access bore into the skull. Depending on the type of procedure, once this initial hole is formed another instrument, a craniotom, is used to cut the skill so that a large portion of the skull can be removed. In some procedures, the bore formed by the perforator provides sufficient access to the underlying tissue on which the remainder of the procedure is to be performed.

During the process of forming the bore in the skull, care must be taken avoid damaging the underlying tissue. In particular, between the brain and skull is the dura. The dura is a fibrous membrane that covers and protects the brain. During a neurological procedure, the dura should be damaged as little as possible so as to ensure it its protective properties are not reduced.

There have been efforts to provide a cranial perforator that, as soon as it forms a bore in the skull, stops advancing forward. This is to minimize, if not eliminate damage to the dura. Many of these perforators include a drive head from which inner and outer drills extend. The inner drill is typically in the form of a cylinder. The outer drill is in the form of a sleeve disposed over the inner drill. The drills are formed with cutting flutes at their distal ends. Typically, a spring is located between the drive head and the inner drill. When the perforator is pressed against the bone, the force the spring places on the drills is overcome. At least one of the drills abuts the drive head. Consequently, the rotation of the drive head results in the like rotation of the drills. The drills are thus rotated and cut the bone. Once one of the drills breaks through the bone, the force of the spring, of at least some perforators, was believed to push the drills away from the drive head. This disengagement of the drills from the drive head causes the drills to stop rotating. This cessation of drill rotation minimizes damage of the underlying dura.

Known perforators are able to form bores in skulls to which they are applied. However, upon boring through the bone, they still engaged in some travel. The displacement of the drills of certain of these perforators is known to potentially expose the underlying dura to injury.

Moreover, care must be taken when initially pressing the perforator against the skull to start to the boring process. The skull is a smooth curved structure. Consequently, the pointed end of the perforator inner drill has been known to slide, to skate, across this surface when the perforator is initially pressed against the bone and actuated. To minimize drill skating, it is known to form the distal end of the perforator inner drill in the shape of a pyramid. This pyramid causes an initial pilot bore to be formed upon the actuation of the perforator. The presence of this pilot bore minimizes skating when additional force is used to press the perforator against the skull.

However, when a perforator is provided with a leading pyramid, the resultant pilot bore is known to fill with bone shavings. These shavings clog the inner drill. Owing to the friction of the cutting process, these shavings can be rather warm. The heat generated by these shavings can potentially damage surrounding tissue that would otherwise not be affected by the bore drilling process.

Moreover, it is desirable to construct the cranial perforator so that, during the process of using it to form a bore, it can be stopped, removed from the bore, reinserted into the bore and restarted. This feature allows the surgeon to, periodically during the bore formation process, inspect the bore. Instructing surgeons find this feature especially useful when training new surgeons.

When a cranial perforator is removed from a partially formed bore, the spring causes the drills to disengage from the drive head. Some known perforators do not easily reset once their drills have so disengaged. Once removed from a partially formed bore, this type of perforator may be difficult to reset and restart in order to complete the formation of the bore.

### SUMMARY OF THE INVENTION

This invention is directed to a new and useful perforator for forming a bore in bone. The perforator of this invention is especially useful for forming a bore in the skull. The perforator of this invention is designed so as that its inner and outer drills stop rotating very shortly after the inner drill penetrates the bone in which the bore is being formed. The perforator of this invention is further designed to minimize the extent to which bone chips accumulate in the pilot bore formed by actuation of the perforator.

The perforator of this invention includes a drive head and inner and outer drills. The perforator is constructed so that, when the inner drill penetrates the bone, the inner drill is driven forward by the caming of the inner drill against the outer drill. This causes the inner drill to disengage from the drive head. The disengagement of the inner drill from the drive head inhibits further actuation of both drills.

The inner drill of this perforator has a number of forward facing cutting flutes. Some, but not all of these flutes, meet at the center of the drill to form a pyramid. When the perforator is pressed against the bone, this pyramid forms a pilot bore. The bone chips formed in this bore are discharged from it through the channels formed in flutes that do not form the pyramid.

The perforator of this invention is also provided with an inner drill with features that minimize the extent to which the inner drill, upon reinsertion into a partially formed bore, penetrates the bone at the base of the bore. This feature as well as the geometry of how the inner drill engages the drive head, increases the likelihood that when the perforator is reinserted in the bore, the drive head will engage and actuate the inner drill so as to rotate the latter component.

### BRIEF DESCRIPTION OF THE DRAWINGS

The invention is pointed out with particularity in the claims. The above and further features and benefits of the perforator of this invention are understood by reference to the Detailed Description below and the accompanying drawings in which:

FIG. 1 is a perspective view of a perforator constructed in accordance with this invention;

FIG. 2 is an exploded view of the perforator;

FIG. 3 is a plan view of the head of the perforator of this invention;

FIG. 4 is a cross sectional view of the perforator head;

FIG. 5 is a perspective view of the drive cap;

FIG. 6 is a cross sectional view of the drive cap;

FIG. 7 is a side view of the plunger;

FIG. 8 is side view, shown in partial cross section, of the inner drill;

FIG. 9 is perspective view of the proximal end of the inner drill;

FIG. 10 is a side view of the inner and outer drills assembled together;

FIG. 10A is a cross sectional view of the inner drill through a plane perpendicular to the longitudinal axis of the drill that is located proximal to the cutting edges of the flutes integral with the drill;

FIG. 10B is an enlarged side view of where the distal edge surfaces of the flutes integral with the inner and outer drills meet;

FIG. 11 is a plan view of the flutes integral with the inner and outer drills;

FIG. 12 is a perspective view of the inner and outer drills;

FIG. 13 is a side view, in partial cross section, of the outer drill,

FIG. 14 is a plan view of the proximal face of the outer drill;

FIG. 15 is plan view of the proximal end of the outer drill showing one of the ramp surfaces of the outer drill against which a complementary leg of the inner drill abuts;

FIG. 16 is a cutaway view showing the relative orientation of the components of the perforator when only the inner drill is engaged for axial loading by the perforator head;

FIG. 17 is a cutaway view showing the relative orientation of the components of the perforator when both the inner and outer drills are engaged for axial loading by the perforator head; an enlarged perspective view of one of the slots formed in the proximal face of the outer drill.

FIG. 18 is a perspective view of the relative orientation of the inner and outer flutes when the inner and outer drills are being rotated to form a bore; and

FIG. 19 is a cutaway view showing the relative orientation of the components of the perforator when the inner drill has, as result of the absence of axial resistance, disengaged from the drive head.

### DETAILED DESCRIPTION

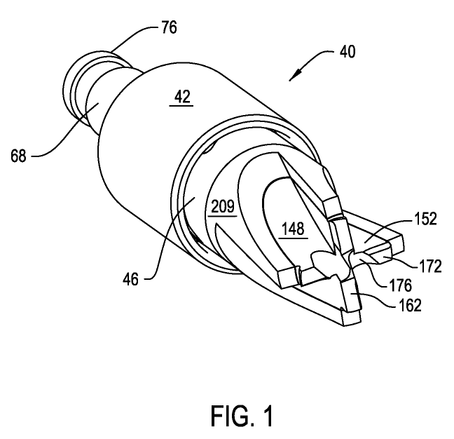

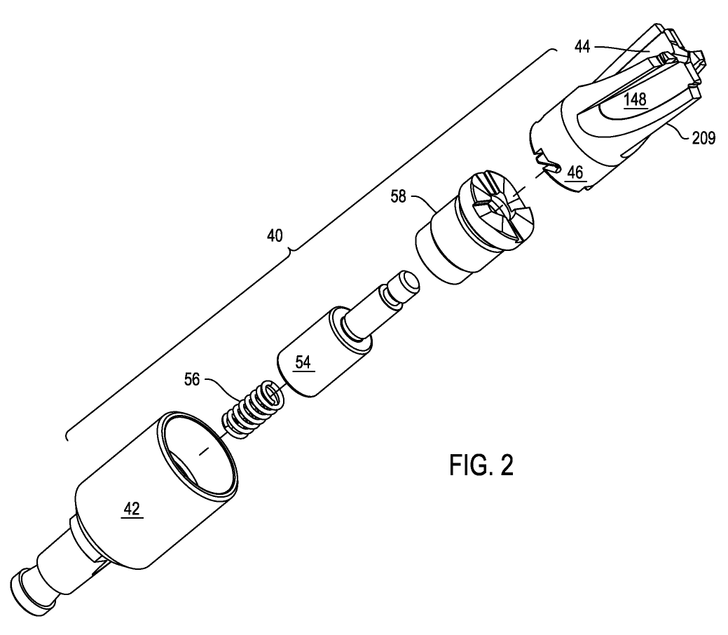

FIGS. 1 and 2 illustrate a perforator 40 constructed in accordance with this invention. Perforator 40 includes a drive head, head 42, from which inner and outer drills 44 and 46, respectively, extend. Inner drill 44 is generally cylindrically shaped. Outer drill 46 is generally tube shaped and disposed over inner drill 44. As discussed in detail below, the inner drill 44 is formed with cutting flutes 146-152. Outer drill 46 is formed with cutting flutes 209.

A plunger 54 disposed inside the head 42 is connected to the inner drill 44. A spring 56, also disposed inside the head 42 abuts the plunger 54. Spring 56 urges the plunger 54 and, by extension, the inner and drill 44 distally forward. (“Distal” is understood to be away from the clinician holding the perforator 40, towards the patient. “Proximal” is understood to mean towards the clinician, away from the patient.) A drive cap 58 is disposed over the distal end of the head 42. Drive cap 58 limits the extent to which the spring 56 can push the plunger out of the head 42. As will be discussed below, the inner drill 44 and drive cap 58 are formed with complementary features. When these features engage, the rotation of the head and drive cap results in the like rotation of the inner and outer drills 44 and 46, respectively.

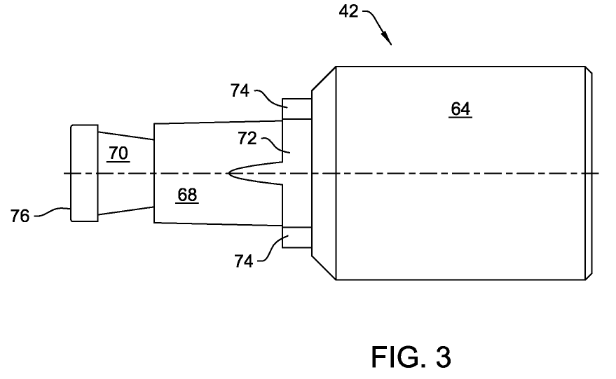

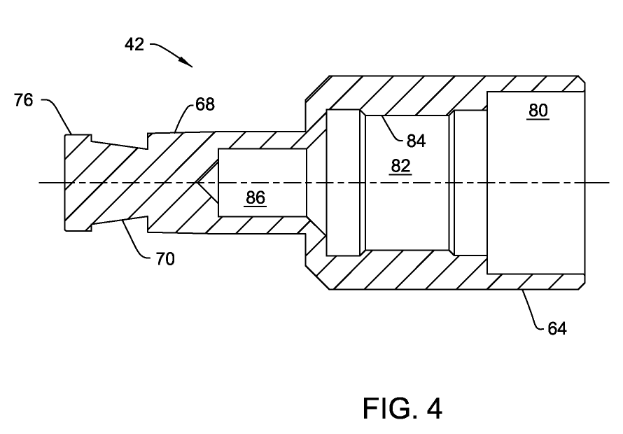

As seen in FIGS. 3 and 4, the perforator head 42 includes a number of concentric, longitudinally aligned sections. At the most distal end is a cylindrical base 64. Base 64 is the largest diameter portion of head 42. Extending proximally rearward from the base 64, there are one or more sections adapted to be secured to and driven by the chuck integral with a drill. The exact type of chuck with which head 42 is configured to be driven is not relevant to this invention. For the purposes of example, head 42 is shown as having features that enable the head to be engaged in and driven by a Hudson chuck. Specifically, extending proximally rearward of base 64, head 42 has first and second stem sections 68 and 70. Stem sections 68 and 70 are concentric with base 64. First stem section 68, the stem section closest to base 64, while generally circular in cross sectional profile, has a diameter that varies. Specifically, the diameter of the first stem section 68 decreases as the section extends proximally rearward from the base 64. The decrease is at angle that is between 0.5 and 5° offset from the longitudinal axis of the stem section 68. In more preferred versions of the invention, this offset angle is between 1 and 2°. Further, head 42 is formed so that stem section 68 is formed with a pair of diametrically opposed, parallel flats 72, one shown. Each flat 72 extends rearwardly from where the stem section 68 extends from the head base 64. Adjacent where the first stem section 68 emerges from the head base 64, there is a pair of wings 74. The end faces of the wings 74 are flat and coplanar with the adjacent flats 72 formed integrally with first stem section 68.

The second stem section 70 extends proximally rearward from the first stem section 68. The second stem section has a frusto-conical shape and is arranged so that the narrow diameter end is the end adjacent the first stem section. A cylindrical cap 76, also part of head 42, is disposed over the proximal end of the second stem section 70. Cap 76 has a diameter greater than that of the adjacent proximal end of the second stem 70.

When head 42 is fitted to a Hudson chuck, balls integral with the chuck abut the tapered surface of the second stem section 70. Since these balls are trapped between the first stem section 68 and the cap 76, both of which that extend beyond the second stem section 70, the balls lock head 42 in the chuck. The chuck also has a pair of planar spaced apart drive plates. When head 42 is seated in the chuck, the plates abut the flats 72 and the end faces of wings 74 coplanar with the flats. The abutment of the drive plates against these surfaces of the head 42 are what transfers the rotational moment of the chuck to the head 42 and, by extension, the rest of the perforator 40.

Head 42 is also formed to have three concentric contiguous bores 80, 82 and 86 that extend inwardly from the distally directed face of head base 64. Bores 80, 82 and 86 are centered along the longitudinal axis of the head 42. Bore 80, the distal most bore, forms a distal end opening into the head base 64. Bore 82 extends proximally from bore 80. Bore 82 has a diameter less than that of bore 80. The perforator head 42 is further formed so that the center slice of the annular wall that defines bore 82 is formed with threading. In FIG. 4, this threading is depicted by ledge 84 that projects inwardly into bore 82. Bore 86 is the most proximal of the head bores. (Not identified is the taper between bores 82 and 84.) Bore 86 has a diameter less than that of bore 82. Bores 80 and 82 extend through the head base 64. Bore 86 extends proximally from bore 82 through head first stem section 68. Bore 86 is, at its proximal end, closed.

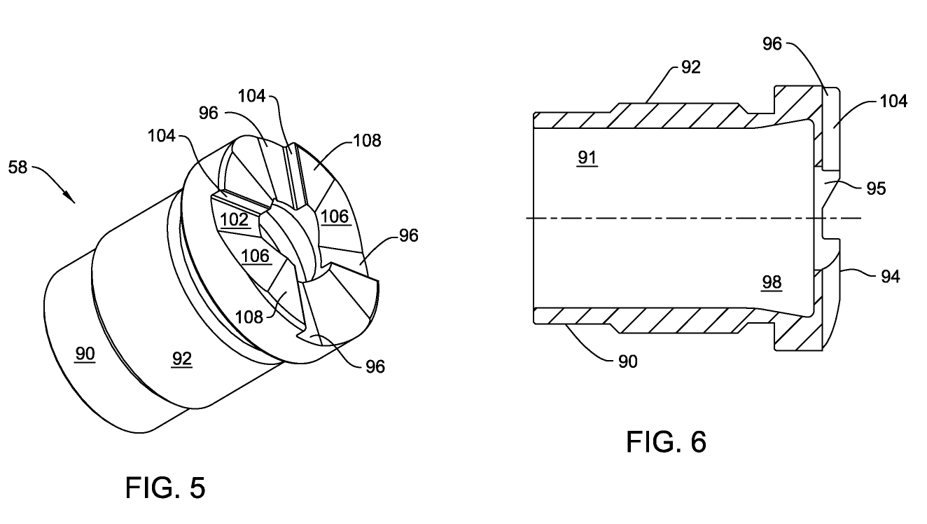

Drive cap 58, now described by reference to FIGS. 5 and 6, is disposed in head bores 80 and 82. The drive cap 58 includes a tube like sleeve 90. Sleeve 90 thus has an inner annular wall 91 that defines a cylindrical void space within cap 58 (void space not identified). The drive cap 58 is formed so that inner wall 91 has a constant diameter and extends from the proximal end of the sleeve 90 substantially the entire length of the sleeve. The outer surface of sleeve 90 is provided with threading represented in the Figures by an elongated annular rib 92 around the outside of the sleeve 90. Sleeve 90 is dimensioned to be fitted in head bore 82 so that complementary threading within the head bore 82 and around the sleeve hold the drive cap 58 in static position within the head 42.

Integrally formed with sleeve 90, the drive cap 58 has a disk shaped end plate 94. The end plate 94 is disposed over the distal end of sleeve 90. While the end plate 94 is generally circular, the drive cap 58 is formed so that the end plate 94 has a center located through hole 95. Drive cap 58 is further formed so that the end plate 94 subtends a circle with a diameter greater than that subtended by sleeve 90.

The drive cap 58 is also constructed so that adjacent the end plate 94, sleeve 90 has a distal inner wall section 98 that extends forward from inner wall 91. Inner wall section 98 is different from inner wall 91 in that, as wall section 98 extends distally forward, the wall section 98 flares outwardly. Inner wall 98 thus defines an undercut in the distal end of the sleeve 90 immediately adjacent end plate 94 (undercut not identified).

Drive cap 58 is further formed so that the outer, distally directed annular face of the end plate 94 has four equangularly spaced apart notches 96. The base of each notch 96 is defined by a base surface 102. A wall 104 extends perpendicularly upward from one end of the base surface 102 to define one end of the notch 96. The opposed end of the notch 96 is defined by a ramp 106. The ramp 106 spirals upwardly away from the base surface with which it is associated. Each ramp 106 terminates at a raised face 108. The raised face 108 terminates at the edge of the wall 104 associated with the adjacent notch 96.

When perforator 40 is assembled, the drive cap 58 is coupled to the head 42 so that end plate 94 is disposed in head bore 80. The abutment of the end plate 94 against the annular step between bores 80 and 82 limits rearward movement of the drive cap 58 in the head 42. More particularly, the components of the perforator 40 are dimensioned so that the outer surfaces of the end plate 94 are proximally rearward of the open end of head bore 80. Thus, within bore 80 there is a void space located forward the distally forward of the drive cap end plate 94.

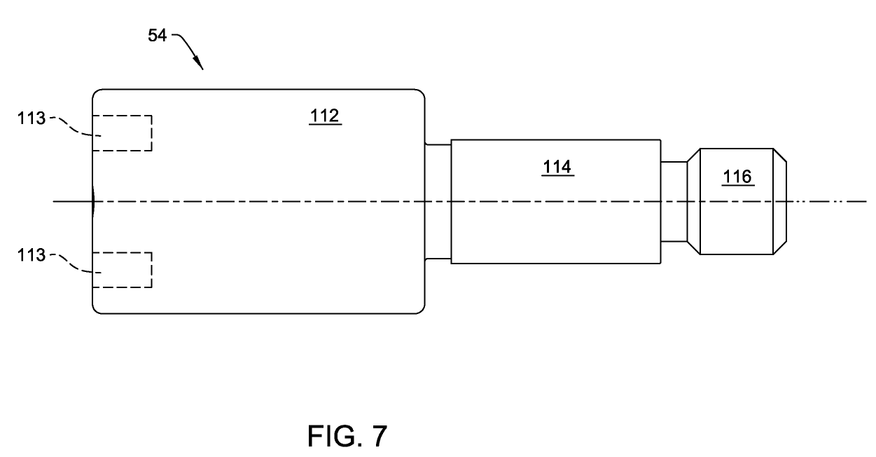

The plunger 54, now described by reference to FIG. 7, is formed from a single piece of metal. A cylindrical head 112 is the most proximal portion of the plunger. Plunger head 112 is dimensioned to closely slip fit in void space defined by drive cap inner wall 91. As seen in phantom, plural closed end bores 113 extend inwardly from the proximally directed face of the plunger head 112. During assembly of the perforator 40, bores 113 receive an insertion tool used to facilitate the screw securement of the plunger 54 to the inner drill 44.

Extending distally from the head 112, the plunger 54 is formed to have coaxial proximal and distal stem sections 114 and 116, respectively. The proximal stem section 114 extends from the distally directed face of the plunger head 112. Stem section 114 extends out of the perforator head 42 through drive cap through hole 95. The proximal stem section 114 has a diameter slightly less than that of the drive cap through hole 95. This dimensioning, as well as the relationship of the plunger head 112 to the void space internal to the drive cap 58, allows the plunger 54 to rotate relative to the perforator head 42 and drive cap 58.

Distal stem section 116 extends forward from stem section 114. Stem section 116 has an outer diameter less than that of stem section 114. The outer circular surface of stem section 116 is provided with threading, (not illustrated). Illustrated by nut identified are the undercut between plunger head 112 and proximal stem section 114 and the undercut between two stem sections 114 and 116.

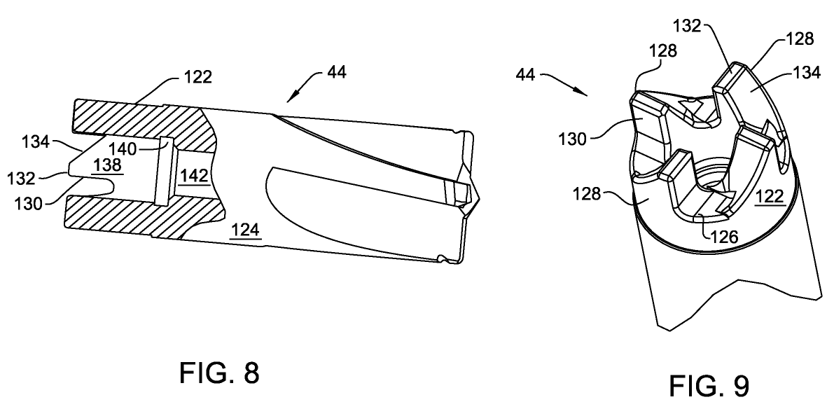

The inner drill 44, initially described by reference to FIGS. 8 and 9, while generally cylindrical, has two coaxial sections 122 and 124, with different diameters. There is a proximal section, section 122 and a distal section, section 124. Proximal section 122 has a diameter slightly less than that of distal section 124. In some versions of the invention, proximal section 122 has a length that comprises from 20 to 40% the overall length of the inner drill 44; the remainder being the distal sectional 124 and the flutes 146-152 integral therewith.

Inner drill proximal section 122 defines a proximally directed face 126. Face 126 is actually divided into four sections by four equangularly spaced apart, proximally extending legs 128. Each leg 128 is shaped to define a first surface 130 that extends perpendicularly away from the adjacent section of the proximally directed face 126. Not identified is the curved transition surface between each face section 126 and the adjacent leg surface 130. Leg surface 130 ends at a leg second surface 132 that is perpendicular to the surface 130. The four leg surfaces 132 thus collectively are the four butt end, proximal end, surfaces of the inner drill 44. Extending downwardly from the leg surface 132 is a third leg surface, ramp 134. Ramp 134 has a slope that is constant between the section of face 126 to the leg surface 132 between which the ramp extends. In some versions of the invention, this angle of the ramp, relative to the longitudinal center axis of the inner drill 44 is between 35 and 50°. In some preferred versions of the invention this angle is between 42 and 44°. Since the slope of ramp 134 is constant, ramp 134 is planar. Mathematically, ramp 134 is a helix.

Inner drill 44 is further formed to have a number of coaxial bore sections that extend distally forward from the proximal end of the drill. A first bore, bore 138, is defined by the inner arcuate surfaces of legs 128 and extends forward from the leg surfaces 130. Bore 138 is dimensioned to closely slip fit receive plunger proximal stem section 114. The bore 138 terminates along the plane that defines the step between inner drill sections 122 and 124. The inner drill 44 is formed so that contiguous with and immediately adjacent bore 138 there is a bore 140. Bore 140 has a diameter that is slightly greater than the diameter of bore 138. Inner drill 44 is formed so that bore 140 is located in the most proximal portion of the drill distal section 124

The inner drill 44 is further formed so that distal to bore 140 there is a bore 142, also in drill proximal section 140. Bore 142 has a diameter less than the diameter of bore 140. Not identified is the taper between bores 140 and 142. The inner annular surface of the inner drill 44 that defines bore 142 is provided with threading, (not illustrated.) Bore 142 and its threading are designed to receive the threaded distal stem section 116 of plunger 54. Thus, upon assembly of the perforator 40, the engagement of stem section 116 in bore 142 locks the inner drill 44 and plunger 54 together. At this time, plunger stem section 114 is seated in inner drill bores 138 and 140. The components are further constructed so that, upon assembly, the inner drill legs 128 are spaced from the adjacent distally directed face of plunger head 112. This gap is sufficient to accommodate, the driver end plate 94, which is disposed around the proximal stem section 114, such that there is a clearance between the end plate and the inner drill legs 126.

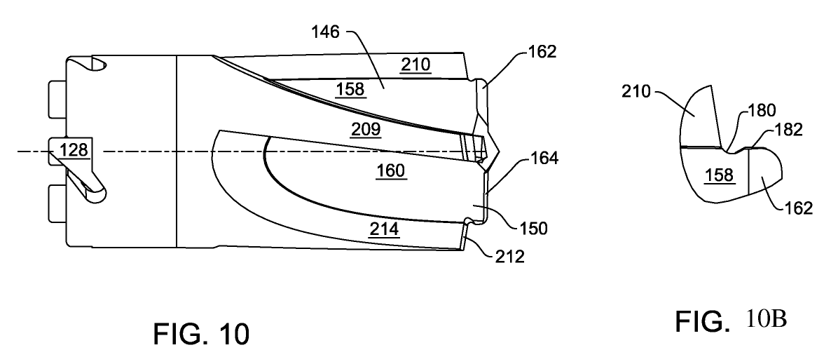

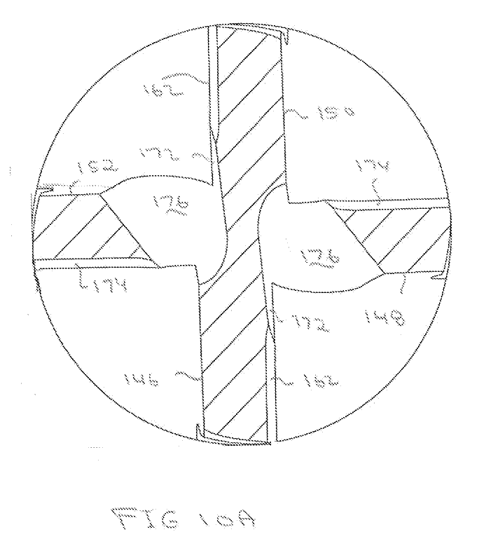

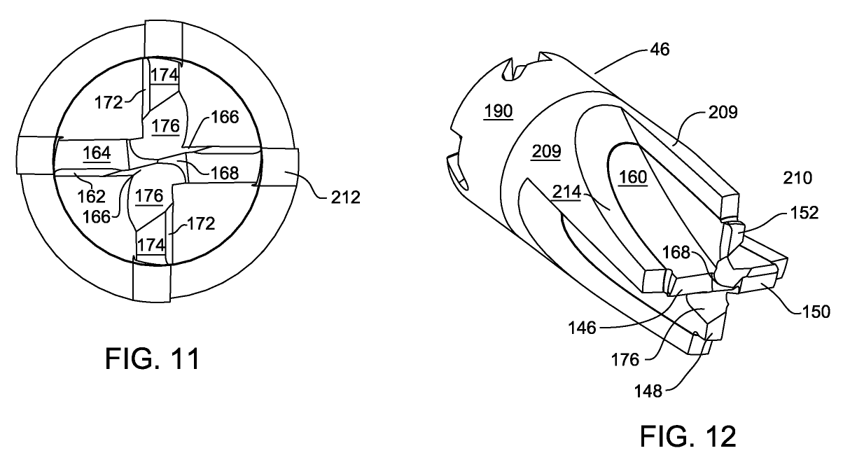

The distal end of the inner drill 44 is now described by reference to FIGS. 10, 11 and 12. Four flutes 146, 148, 150 and 152 extend forward from the solid cylindrical core of the inner drill distal section 124 to form the distal most portion of the drill 44. Each flute 146, 148, 150 and 152 is formed to have opposed forward and trailing surfaces 158 and 160, respectively. The flutes 146-152 are formed so that, extending from where the faces 158 and 160 emerge, surfaces 158 and 160 curve forward, in the direction of the rotation of the drill 44. Flutes 146-152 are equangularly spaced apart from each other. Flute 146 is longitudinally aligned with and symmetric with flute 150. Flute 148 is longitudinally aligned with and symmetric with flute 152.

Flutes 146 and 150 each have a first cutting face 162 and a first flank surface 164. Each first cutting face 162 extends distally from the associated flute forward surface 158 and is angled slightly rearwardly from the associated forward surface. This angle is between 20 and 30° relative to the longitudinal axis of the perforator. In some preferred version of the invention, this angle is between 23 to 27° relative to the longitudinal axis of the perforator 30. The first flank surface 164 is contiguous with each first cutting face 162 and extends rearward, opposite the direction of drill rotation, from the cutting face. Each first flank surface 164 lies on a plane that that is offset from the longitudinal axis of the perforator by no more than 88°. In some versions of the invention, the maximum offset of the first flank surfaces is no more than 82° from the longitudinal axis of the perforator. The longitudinal axis of each first flank surface 164, the axis that extends from the outer perimeter of the inner drill 44 towards the center is generally perpendicular to the longitudinal axis of the drill 44. The edges along which each pair of first cutting faces 162 and first flank surfaces 164 meet form a first set of cutting edges of the inner drill 44 (edges not identified). The trialing edge of each first flank surface 164 abuts the distal edge of the associated flute trailing surface 160.

Flutes 146 and 150 also each have a second cutting face 166 and second flank surface 168. Relative to the outer perimeter of the inner drill 44, each second cutting face 166 is located immediately inward of the adjacent first cutting face 162. Each second cutting face 166, extends upwardly and rearwardly from the associated flute forward surface 158. The rearward angle of each second cutting face 166 is less than that of the adjacent first cutting face 162. Each second flank surface 168 extends rearwardly relative to the second cutting face 166 with which the surface abuts. Each second flank surface 168 lies in a plane that is between 15 and 45° offset from the plane of the adjacent first flank surface 164. In some preferred versions of the invention, each second flank surface 168 lies in a plane that is between 25 and 35° offset from the adjacent first flank surface.

The opposed flute second flank surfaces 168 of flutes 146 and 150 rise and meet at the center of the drill. Collectively, the flute second flank surfaces 168, thus define a pyramid, (not identified). This pyramid projects above the outer portions of flutes 146 and 150, the portions of these flutes below the first flank surfaces 164. The apex of this pyramid is the edge along which the opposed second flank surfaces 168 meet. In some versions of the invention, the inner drill 44 is shaped so that apex of the pyramid, the edge along which the second flank surfaces 168 meet, has a length of 0.030 inches or less. In some preferred versions of the invention, this length is 0.020 inches or less. In more preferred versions of the invention, this length is 0.010 inches (0.025 cm) or less.

The edge along which each second cutting face 166 and associated flank surface 168 meet form a cutting edge (not identified). Thus, the pyramid is formed to have two cutting edges that are reverse symmetric around the longitudinal axis of the inner drill 44.

Flutes 148 and 152 each have a cutting face 172 and a flank surface 174. Geometrically, cutting faces 172 are at identical angles to the first cutting faces 162 of flutes 146 and 150. Flank surfaces 174 are identical to the first flank surfaces 164 of flutes 146 and 150. Cutting faces 162 and 172 are thus angled rearwardly away from forward surfaces 158 of the flutes from which the cutting surfaces extend. This angle provides flutes 146-152 with a negative rake.

Each flute 148 and 152 is further formed to have a concave face 176. Each face 176 is located adjacent the inner termini of the associated cutting face 172 and flank surface 174, close to the longitudinal axis of the inner drill 44. Flutes 146-152 are further formed so that each face 176 merges into the second cutting face 166 of a first one of the adjacent flutes 146 or 150. Flutes 148 and 152 are further formed so that the associated face 176 extends across the width of the flute. Also, each face 176 extends into the second of the adjacent flutes 150 or 146 so as intersect the second flank surface 168 of the second adjacent flute 150 or 146. The flutes 148 and 150 are further formed so that the radius of curvature of its face 176 has a longitudinal axis that is angled such that the edge of each face abutting the flute trailing surface 160 is proximal to the edge the face forms with the complementary flute forward surface 158. Each face 176 thus forms a channel in the flute 148 or 152 in which the face is formed, (channel not identified).

Inner drill 44 is further formed so that, collectively the second cutting faces 166 of flutes 146 and 150 and the faces 176 of flutes 148 and 152 provide the center pyramid with a tapered profile. That is, progressing downwardly from the apex of the pyramid where flank surfaces 168 meet, the side-to-side width of the pyramid, the width along the axis perpendicular to flank surfaces 168, increases.

Still another feature of flutes 146-152 is that flank surfaces 164 and 174 have a minimum width, from cutting surface to flute trailing surface, of 0.040 inches. In some versions of the invention, this minimum width is 0.050 inches or more. In other versions of the invention, this width is 0.055 inches or more. Also, it should be appreciated that the angle between cutting faces 162 and 172 and, respectively, flank surfaces 164 and 174 is typically at least 70°, in more preferred versions of the invention, this angle is at least 90° and in other versions of the invention, at least 100°.

It should be appreciated that the inner drill flutes 146-152 are formed so that, in a plane perpendicular to the longitudinal axis of the inner drill that is immediately proximal to flute cutting edges, the flutes, including the portions of that define the center pyramid, subtend a relatively large cross-sectional area of the circle defined by the flutes.

Diagrammatically, this is seen in FIG. 10A. Here circle 178, is the circle defined by the outer perimeter of the flutes at a location proximal to their cutting edges. The flutes 146-152 are shown in cross section within circle 178. In many versions of this invention, when this plane is located 0.010 inches proximal to the cutting edges of the flutes 146-158, the flutes subtend at least 10% of the area of the circle they define in this plane. In still other versions of the invention, the flutes subtend at least 15% of the area of this circle. In still other preferred versions of the invention, the flutes subtend at least 20% of the area of this circle. It should be understood that the flute “cutting edges” from which this plane is referenced are the defined by the first cutting edges of flutes 146 and 150, the cutting edges integral with the first cutting surfaces 162, and the companion cutting edges defined by cutting surfaces 172 of flutes 148 and 152. The significance of the flutes 146-152 subtending this amount of the area of the circle they define is discussed below.

As seen in FIG. 10B, flutes 146-152 are further formed so that the outer ends thereof, the ends adjacent the outer drill flutes 209, are rounded. Specifically, the outer end of each flute 146-152 is formed with two contiguous side surfaces 180 and 182 that extend between the opposed leading and trailing surfaces 158 and 160, respectively, of the flute. The proximal of the two side surfaces, surface 180, has a concave profile such that the surface curves inwardly from the outer perimeter of the proximally adjacent section of the flute 146, 148, 150 or 152. At the edge where the flute forward surface 158 meets the flank surface 162 or 166, surface 180 transfers into surface 182. The surface 182 has a convex profile. Each surface 182, as it curves outwardly, merges into the adjacent flank surface 164 or 174 of the flute 146, 148, 150 or 152 with which the surface 182 is integral.

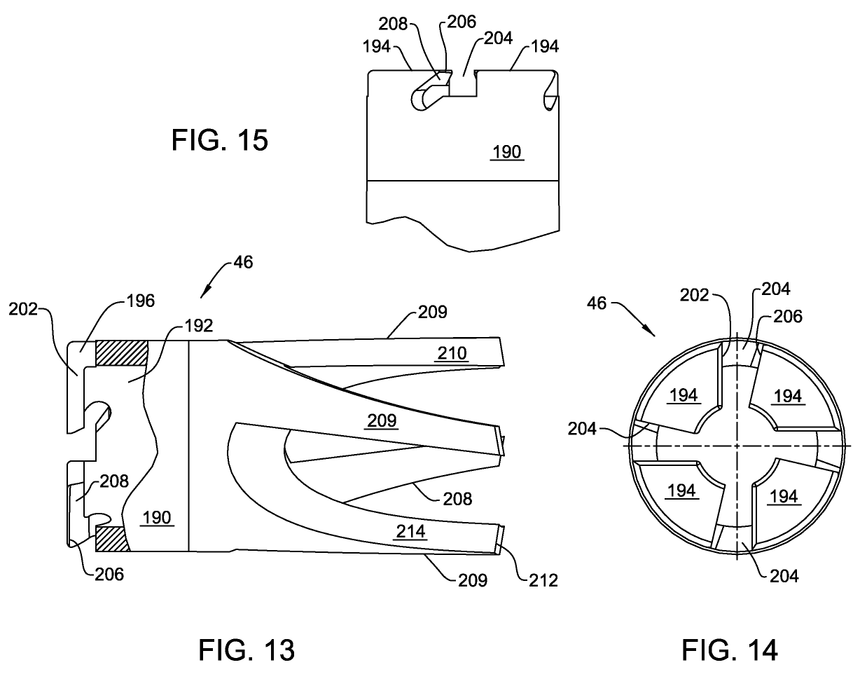

Outer drill 46 is now initially described by reference to FIGS. 13 and 14. The outer drill 46 is formed to have a generally tubularly shaped crown 190 that defines a center bore 192. Crown 190 has an outer diameter dimensioned to allow the outer drill to be slip fitted in drive head bore 80. The outer drill crown 190 is also formed so that inner drill 44 can closely slip fit in bore 192. The distal end of bore 192 is open. Inner drill 44 thus extends out through the distal end of bore 192.

The outer drill 46 is further shaped to have four arcuately spaced apart tabs 194 integrally formed with crown 190 that extend over the proximal end of bore 192. Each tab 194 is generally in the form of an arch with concentric inner and outer radii that are centered around the longitudinal center axis of the drill 46. Integral with each tab 194 is a bracket 196 that extends perpendicularly forward from the plane of the tab, (one bracket shown in FIG. 13). Each bracket 196 serves as the structural component of the outer drill 46 that connects the associated tab 194 to the drill crown 190. Collectively, tabs 194 and bracket 196 are shaped so that the outer circumference collectively subtended by the four tab and bracket pairs is slightly less than the outer circumference of the drill crown 190. In one version of the invention, wherein the crown 192 has an outer diameter of 0.531 inches, (1.35 cm) the circle subtended by the tab and bracket pairs has a circumference of 0.518 inches (1.32 cm).

Each tab 194 is formed to have a leading face 202 and a trailing face 206 that extend forward from the proximal end face of the tab. Thus, the leading face 202 of a first tab and the trailing face 206 of an adjacent second tab define a slot 204 between the adjacent tabs 194. Slots 204 are arranged in opposed pairs. Each tab 194 is shaped so that its leading surface 202 is along a line that is parallel to a radial line extending from the center of the slot 204 defined by the surface 202 and the center of the drill 46. Each tab trailing surface 206 is located along a line offset from a radial line that extends from the center axis of the drill 46. More specifically, there is a radial line that extends from the center axis of the inner drill to the outer edge of the tab trailing face 206. The trailing face 206 is located along a line that, relative to this radial line, is angled forward, towards the lead face 202 of the tab 194. Collectively, tabs 194 are thus arranged so that any two tabs that are 180° opposite each other are mirror images of each other.

Each tab 194 is further constructed so as to have ramp surface 208, best seen in FIGS. 13 and 15, that extends diagonally from trailing surface 206. More specifically, the each ramp surface 208 relative to the proximally directed exposed face of the tab 194, extends both towards the side of the face defining the tab leading surface 202 and distally forward. Each ramp surface 208 extends along an angle of between 48 and 58° relative to the longitudinal axis of the perforator 30. A slot, not identified extends inwardly from the side of the tab bracket 196 adjacent the ramp surface 208. This slot is formed as a consequence of the formation of ramp surface 208 and is not otherwise relevant to this invention. As a consequence of the formation of the ramp surface 208, it should be understood that the tab trailing surface 206 has a very short length, often less than 0.012 inches.

Outer drill 46 is further formed so that four arcuately spaced apart flutes 209, best seen in FIGS. 12 and 13, extend forward from crown 190. The outer drill 46 is formed so that extending distally forward from the crown 190, the diameter of the circle defined by the flutes 209 slightly increases. In some versions of this invention, this outward taper is at least 0.5° relative to the longitudinal axis of the perforator 30. The inner arcuate surfaces of flutes 209 (surfaces not identified, define a space in which the inner drill 44 can be disposed. Each flute 209 has a cutting face 210 and, opposite the cutting face 210, a back surface 214. At the distal end of the flute 209, a flank surface 212 extends between the cutting face 210 and the back surface 214. The edge between each cutting face-flank surface pair is the cutting edge of the flute 209. The angle between these two surfaces is less than 90°. Flutes 209 are further formed to curve forward from where they extend forward from the crown 190. As a consequence of this curvature, the flutes 209 present a positive rake angle. In one version of the invention, each flute 209 is formed so that the cutting face 210 is a planar face that angles forward; the opposed trailing face 214 curves forwardly.

As part of the process of constructing the perforator 40 of this invention, the inner and outer drills 44 and 46, respectively, are partially formed together. Specifically, the proximal ends of these components are first formed in separate machining operations. Thus, in one set of machining operations the inner drill legs 128 and bores 138, 140 and 142 are formed. The outer drill 46 is formed to define tabs 194. At this step of the process, the inner drill still includes a long cylindrical section forward of the bores 138-142; the outer drill is basically a tubular structure. The partially-formed inner drill 44 is then fit into center bore of the partially assembled outer drill 46. More particularly, the drills are arranged so that the ramps surfaces 134 of the inner drill legs 128 abut the ramp surfaces 208 of the outer drill tabs 194 and leg surfaces 130 abut tab surfaces 202. At this time the two partially assembled drills are locked in a fixture. Flutes 146-152 and 209 are simultaneously formed on the respective drills 44 and 46.

This process ensures that cutting edges of the individual drills 44 and 46 will be properly aligned relative to each other. Thus when the drill is in operation the inner terminal points of the cutting edges formed on the outer drill flutes 209 will be in the same plane as the terminal points where curves 180 of flutes 146-152 start to extend inwardly. As discussed below, during operation of the perforator, while flutes 146-152 and 209 are longitudinally aligned, they are not similarly radially aligned.

Perforator 40 of this invention is assembled by placing drive cap 58 around the plunger 54. More particularly, drive cap 58 is positioned so that the cap sleeve 90 is disposed around the plunger head 112 and plunger stem section 114 extends through hole 95 in the cap end plate 64. The inner drill 44, with outer drill 46 fitted thereover, is then screw secured over the plunger stems sections 114 and 116.

Spring 56, which is a coil spring, is disposed inside bore 86 internal to perforator head 42. The spring 56 is of sufficient length so that, when seated in bore 86, the distal end of the spring extends into bore 82. The plunger-drive cap-drill sub-assembly is then attached to the head 42. This operation is accomplished by inserting the plunger 54 and drive cap 56 in head bores 80 and 82 so that the drive cap can be threadedly secured in perforator bore 82. More particularly, the drive cap 56 is secured into bore 80 until the annular outer face of the cap end plate 94 abuts the annular step in the plunger head between bores 80 and 82. As a result of this positioning it should be appreciated that the proximal end of the plunger head 112 bears against and compresses spring 56. Once this process is completed, the perforator 40 is considered assembled.

Prior to use, the drill bits 44 and 46 are not subjected to any axial loading. Accordingly, the force spring 56 imposes against the plunger 54 urges the plunger and, by extension, the inner drill 44, distally forward. This displacement of the inner drill 46 away from the perforator head 42 is sufficient to result in a like displacement of the inner drill legs 128 away from end plate 94.

While spring 56 causes the distal face of the plunger head 112 to abut the adjacent proximally directed face of the end plate 94, there is a limit to the force imposed by the spring. Specifically, the force of the spring 56 is sufficient to hold the inner drill 44 out of engagement with the end plate 94. However, the force of spring 56 is insufficient to generate a substantial drag torque between the distally directed face of the plunger head and the adjacent proximally directed surface of the end plate 94. This allows the perforator head 42 to rotate relative to the plunger-and-drill assembly.

The perforator 40 is readied for use by positioning the pyramid formed by inner drill flutes 146 and 150 against the bone where the bore is to be formed. The perforator is further forced downwardly so as to overcome the force imposed by the spring 56 on the plunger-and-drill assembly. This action results in the drive cap end plate 94 being pressed towards the inner drill legs 128). There is some possibility that, as a result of this relative displacement of the inner drill 44 and end plate 94, the drill legs 128 seat in the cap notches 96. Most likely, the leg surfaces 132 will abut either the drive cap ramps 106 or raised surfaces 108.

Once the perforator 40 is so positioned, the drive unit, the handpiece, that rotates the chuck is actuated. The actuation of the handpiece chuck results in rotation of the perforator head 42. In the event the inner drill legs 128 are not disposed in the drive cap notches 96, there is essentially no transfer of torque from the head-drive cap sub-assembly to the inner drill. At this time, the inner drill flute pyramid is exposed to the resistance of the bone against which the pyramid abuts. This resistance blocks rotation of the inner drill 44. Thus, at this time, the combination of the axial load placed on the head 42, the rotation of the head 42 and the static state of the inner drill 44, results in the movement of the head and drive cap so that cap ramps 106 slide over the inner drill legs 128. This displacement of the perforator head 42 and drive cap 58 continues until the inner drill legs 128 seat against the base surfaces 102 of cap notches 96.

During these steps of setting up the perforator 40 for operation and initially actuating the perforator, outer drill 46 is able to move between the inner drill legs 128 and the distally directed faces 106 of the drive cap end plate 94. Gravity may cause the outer drill 46 to abut the inner drill 44 so that the ramp surfaces 208 of the outer drill tabs 194 seat against the adjacent ramp surface 134 of the inner drill legs 128. During this part of the process, there are no axial forces causing the outer drill flutes 209 to bear against the adjacent bone.

Once the inner drill legs 128 seat in the drive cap notches 96, the continued rotation of the perforator head and drive cap results in the drive cap walls 104 abutting the surface 130 of the inner drill legs 128. The abutments of these surfaces, serves to transfer torque from the perforator head 42 to the inner drill 44. These two components rotate in unison. The combination of this torque and the axial load placed on the inner drill flutes 146-152 results in the cutting edges of these flutes cutting the bone so as to form a bore.

Initially, this cutting process is performed only by the cutting edges formed by the pyramid defined by the second cutting faces 166 and second flank surfaces 168. Thus, this pyramid forms a small pilot bore in the bone. The formation of this pilot bore retains this center located pyramid. The retention of this pyramid substantially eliminates skating of the inner drill during the initial portion of the bore formation process.

During the process of the formation of the pilot bore, heads of bone chips form in front of the cutting surfaces of the pyramid. These bone chips are ejected out of the pilot bore by the discharge channels formed by flute faces 176. The discharge of bone chips out of the pilot bore reduce the extent these chips, during the continued advancement of the perforator 40, clog the pilot bore.

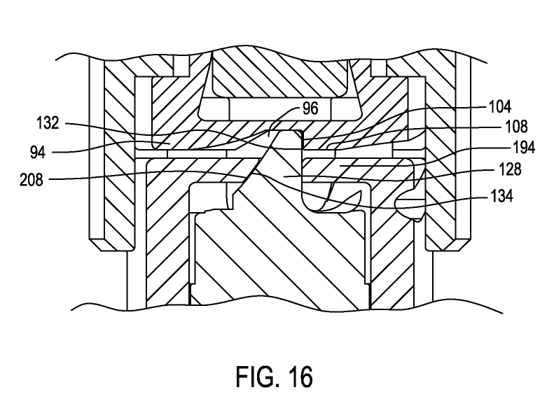

As a consequence of the rotation of the inner drill 44, the inner drill ramp surfaces 134 invariably abut the adjacent ramp surfaces 208 integral with the outer drill 46. However, during the initial process of forming the bore in the bone, the drive cap end plate 94 remains spaced from the outer drill tabs 194 as shown in FIG. 16. Therefore, the outer drill 46 is not subjected to any axial loading. Accordingly, at this stage in the bore formation process, the outer drill flutes 209 may only abut the bone. Since the outer drill flutes 209 are not pressed against the bone, even though they are rotating, in this stage of the process, they do not cut the bone.

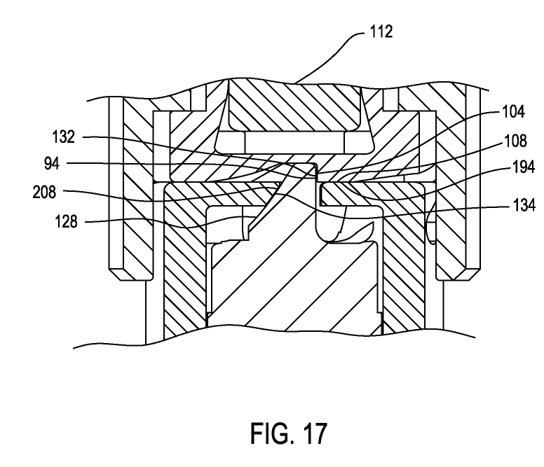

As the process of the bore being formed in the bone by the inner drill 44 continues, the perforator head 42 and drive cap 58 advance toward the outer drill 46. Eventually, the drive cap 58 advances towards the outer drill 46 a sufficient distance so that the cap faces 108 abut the outer surfaces of the outer drill tabs 194 as seen in FIG. 17. The abutment of these surfaces results in the transfer of some of the axial force applied to the perforator head 42 to the outer drill 46. Outer drill 209 flutes are thus forced against bone. Since flutes 209 are rotating, the combined axial load and torque result in the cutting edges of the flutes forming a counter bore around the bore formed by the inner drill flutes 146-152.

From FIG. 17 it can further be observed that as a consequence of the dimensioning of the components of perforator 40, when the outer drill tabs 194 abut the distal face of end plate 94, the tabs are spaced proximally from proximally directed face 126 of inner drill 44. Also, outer drill 46 is formed so that when the inner drill legs 128 seat in end plate notches 96, there is a clearance between the inner drill leg surface 130 and the adjacent outer drill tab leading surface 202. Thus, outer drill 46 is formed so that there is sufficient clearance in slots 204 for the inner drill legs 128 to fully seat in the end plate notches 96 and for there to be a small play in between the legs 128 and surrounding outer drill tabs 194. Generally, the radial separation between surfaces 130 and 202 when the legs abut the bases of notches 96 is a minimum of 0.5° and in some versions of the invention 2° or more.

During this simultaneous rotation and axial loading of the inner and outer drills, the inner drill 44 is exposed to greater cutting torque from the bone being cut than the cutting torque to which outer drill 46 is exposed. This is due to the different rake angles of flutes 146-152 and flutes 209. This is also due to the difference in angles around the cutting edges of flutes 146-152 and flutes 209. In other words, the angle between cutting faces 162 and 172 and, respectively, flank surfaces 164 and 174 is greater then the angle between outer flute cutting faces 209 and the adjacent flank surfaces 212. Therefore, more torque is applied to the inner drill 44 than the outer drill 46.

As a consequence of this difference in torque, the disengaging force applied to the inner drill 44 due to the cutting torque of the outer drill 46 is less than the engaging force imposed on inner drill 44 due to the axial loading of the inner drill 44. The difference in these forces means that as the drills 44 and 46 continue to rotate, the inner drill legs 128 remain seated in the drive cap notches 96 against the surfaces 202. Therefore, the rotational moment of the head and drive cap is continued to be transferred to the inner drill and, through the inner drill 44 to the outer drill 46.

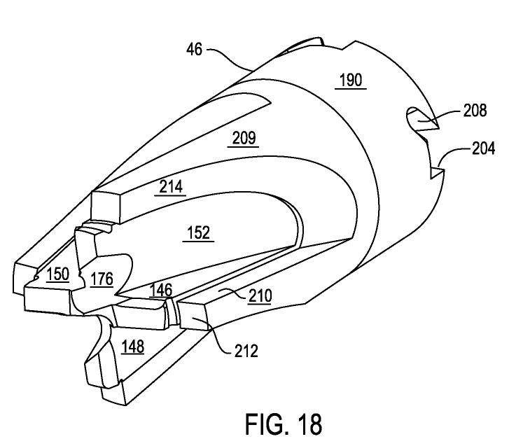

As a consequence of the geometric arrangement of the inner drill legs 128 and outer drill tabs 194, when the inner and outer drills are simultaneously rotated, there is a slight shifting in rotation alignment of the drills relative to each other. Due to this shift, the inner drill flutes 146-152 and surrounding outer drill flutes 209 likewise go out of alignment. Specifically, the outer drill 46 shifts relative to the inner drill 44 so that each outer drill flute 209 shifts approximately 4° of the adjacent inner flute 146, 148, 150 or 152 as seen in FIG. 18. The actual offset is directly proportional to the above-described radial separation between surfaces 130 and 202.

As a consequence of the above described relative positioning of flutes 146-152 and flutes 209, the bone chips formed by flutes 146-152 are not immediately discharged into the path of flutes 209. Instead, the bone chips formed by flutes 146-152 are discharged in front of the head of chips formed by the flutes 209. This minimizes the clogging of the flutes 209.

During the process of forming the bore, the perforator 40 may be subjected to side loading. “Side loading” is understood to be the application of longitudinal force towards the bone at an angle to longitudinal axis of the perforator 40. In this side loading occurs, the plunger head 112 may become axially offset relative to the longitudinal axis of the perforator head 42. In the event such displacement occurs, the outer circumference of the plunger head 112 enters the annular undercut void space defined by drive cap inner wall 98. This void space is seen in FIG. 17. The entry of the plunger head 112 into this undercut substantially eliminates the likelihood that, during such side loading, the plunger could abut the drive head inner wall. If such abutment is allowed to occur, the resultant wear could cause the plunger to stick to the head 42. Such sticking would inhibit the ability of the plunger-inner drill assembly to move distally relative to the perforator head 42.

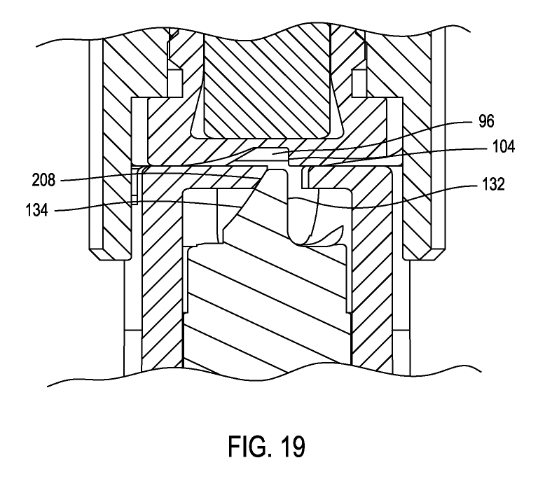

Eventually, inner drill flutes 146-152 cut through the bone in which the bore is being formed. Since the outer drill flutes 209 are proximally rearward of the inner drill flutes 146-152, the outer drill flutes 209 remain embedded in the bone. At this time, the resistive and torque loads the bone places on the inner drill 44 essentially falls to zero. The inner drill 44 still receives the torque transmitted by the perforator head 42 and drive cap 58 to the drill legs 128. However, the bone is still placing a resistance on the rotation of the outer drill 46. Further, at this time, the full axial load supplied by the practitioner is fully transferred through the outer drill 46 to the bone. Owing to this difference in torque and axial loading and the angled abutment of the inner drill ramps 134 against the outer drill ramps 208, the torque applied to the inner drill legs is converted into an axial force that urges the inner drill 44 distally, away from the drive cap. Eventually, as illustrated by FIG. 19, the inner drill is displaced to the point at which the drill legs 128 extend completely away from the endplate notches 96. When this event occurs, the inner drill 44 is no longer the recipient of any torque from the perforator head 42 and drive cap 58. Therefore, by extension, the inner drill 44 stops transmitting torque through ramp surfaces 134 and 208 to the outer drill 46. Accordingly, owing to the resistance the bone places on the outer drill flutes 209 in opposition to their rotation, the outer drill also stops rotating. The cessation of outer drill 46 rotation blocks further rotation of the inner drill 44. The inhibiting of the rotation of the inner drill 44 also results in a like cessation of its axial advancement.

Accordingly, it has been found that once the inner drill penetrates the bone and starts to retract from the drive cap 58, the inner drill rotates less than 20°, usually less than 15° and, often 10° or less before both drills 44 and 46 stop rotating.

Further, another feature of perforator 40 is that, should the inner drill 44 press against the dura, the outer surfaces of the drill that come into contact are the curved surfaces 180 and 182. Thus, owing to the fact that these surfaces, as they extend outwardly, curve inwardly, they do not expose the dura to sharp edges. This minimizes the likelihood that should the flutes 146-152 when so pressed or rotated against the dura will appreciably damage this tissue.

As discussed above, there are relatively blunt angle around the cutting edges of the inner drill flutes 146-152 and flank surfaces 164 and 174 are of relatively large width. These features in combination with the number of flutes provided mean that immediately proximal to the inner drill flute cutting edge, these flutes have a cross sectional area that occupies a relatively large percentage of the area of the circle defined by these flutes. This circle, circle 178 of FIG. 10A, defines the bore formed by the inner drill 44. The above features increase the likelihood that, when perforator 40 is removed from a partially completed bore, reinserted in the bore and restarted, drills 44 and 46 will reengage engage the perforator head 42. Specifically, when the perforator 40 is reinserted in the bore, the surgeon applies axial force to the inner drill flutes 146-152. However, owing to wide surface area over which the this force is applied and the bluntness around the cutting edges of the flutes 146-152, the force per unit area, the pressure, applied to the cutting edges and adjacent surfaces is, in many situations, not sufficient to significantly overcome the resistance to deformation the underlying bone imposes in opposition to this pressure. This is believed to be true even when the flutes are pressed against relatively soft, porous cancellous bone.

This minimal penetration of the bone by the inner drill flutes 146-152 is significant if, during the process of resetting the perforator in the bore, the inner drill is positioned such that its legs 128 are not seated in drive cap notches 96. This can happen if the axial force imposed on the inner drill 44 causes its flutes to sink in bone and the outer drill flutes 209 merely rest on the annular step previously formed by these flutes 209. If the perforator is so positioned, and the drive cap ramps 106 are not present, the perforator could be in a state wherein both the inner drill legs 128 and outer drill tabs 194 seat against the distally directed face of the drive cap end plate 94. If the bone against which the outer drill 46 is pressed is so dense that it does not allow the outer drill through axial force alone, penetrate into the bone, the outer drill 46 may function as a support pylon that blocks the drive cap from moving forward over the inner drill legs 128. In this event, even when the drive cap notches 96 are rotated so as to come into registration with the slots 204, due to the blocking effect of the outer drill 46, the end plate 94 will not seat over the inner drill legs 128. Should the end plate 94 and inner drill legs 128 so fail to engage, the head and drive cap assembly will not transfer torque to the inner drill 44.

Instead, with perforator 40 of this invention, the geometry of the flutes 146-152 limits the extent that, even when subjected to significant manual axial loading, the flutes can be pushed into the bone. Also, the actual percent of the surface of the distally directed faces of end plate 94 occupied by the raised faces 106 is less than 40% of the overall surface of the end plate against which the legs 128 of the inner drill can abut. In some versions of the invention, the percentage of surface area occupied by these faces is less than 35% of the potential surface area of the legs 128 could abut. In other preferred versions of the invention, the surface area occupied by raised surfaces 106 is less than 30% of the surface area that legs 128 could abut. Should the perforator be repositioned in the partially formed bore such that the inner drill legs 128 are seated against the raised faces 108 the following sequence of events will occur: (1) The axial load the surgeon applies to the perforator head is applied to the inner drill 44. However, owing to the blunt profile of the distal end of flutes 146-152 and the distribution of the axial load over a wide area, the forward movement of the flutes 146-152 into the bone is limited. Consequently, the outer drill 46 does not function as a support pylon that blocks the distal movement of the plunger head and drive cap. (2) The perforator head 42 and drive cap 58 are rotated while the axial force is applied. Again, the forward axial displacement of the inner drill 44 is limited. (3) The rotation of the drive cap results in the inner drill legs 128 bearing against the drive cap ramps 108. (4) Consequently, the continued axial force applied by the surgeon to the perforator head 42 results, during this rotation of the end plate 94, the end plate being displaced forwardly over the legs 128 of the inner drill 44. (5) The end plate continues to so rotate until the inner drill legs 128 seat in the end plate notches 96. At this time, the rotational abutment of end plate walls 104 against the inner drill legs 128 results in the transfer of torque to the inner drill 44. The inner and outer drills 44 and 46, respectively, will rotate together as previously described.

It should be appreciated that the above transfer of torque occurs almost immediately after the inner drill legs 128 enter the drive cap notch 94. There is no need for the drill legs 128 to fully abut the drive cap base surfaces 102. This is because all but the least minimal surface contact between drive cap surfaces 202 and inner drill surfaces 132 results in the transfer of torque between the drive cap 58 and the inner drill 44.

Moreover, in the event the perforator head 42 and drive cap 58 are inadvertently rotated in the reverse direction, from left to right in FIG. 19, the drive cap wall 104 does not abut the inner leg. Instead, ramp 106 abuts the adjacent ramp 134 of the leg. The continued rotation of the drive cap 58 results in the rotation moment of the drive cap being transferred into an axial force against the legs 128. This force urges the legs distally forward so they extend away from and are disconnected from the drive cap 58. Thus, in the event the perforator head 42 is inadvertently rotated in the reverse direction, within less than 90° and, in preferred versions of the invention less than 60° of the rotation, the inner drill is disengaged from the perforator head. This substantially eliminates the likelihood that reverse rotation of the drills 44 and 46 and the potential for damage caused by such displacement.

It should be understood that the foregoing is directed to one such version of the invention. Alternative versions of the invention may have features different from what has been described. For example, there is no requirement that in all versions of the invention each of the foregoing features be present.

Thus, in some perforators of this invention, the outer drill may be replaced by a sleeve. This sleeve includes the surfaces that cause the inner drill 44 to disengage from the perforator head 42.

Also, while in the described version of the invention, the inner drill 44 is provided with four (4) flutes, other versions of the invention may have fewer or more flutes. In preferred versions of the invention, however, there are at least four (4) flutes, there is an even number of flutes and the flutes are symmetrically arranged. Also, as discussed above, in the preferred version of the invention, only two of flutes meet to define the center pyramid. The remaining flutes stop short of the pyramid. Thus, the gaps between remaining flutes and the pyramid function as discharge paths through which bone chips formed in the pilot bore by the pyramid are discharged.

Similarly, other features may be present in alternative versions of the invention. For example in order to minimize, if not eliminate, torque transfer to the inner drill 44 when the legs 128 are not seated in the notches 96 other features than ramps are possible. For example, in some versions of the invention, the legs and/or end plate may be coated with material have a very low coefficient of friction. This coating would substantially reduce the friction coupling and therefore the possibility of torque transfer between the perforator head 42 and the inner drill when the inner drill legs are not seated in notches 96.

Likewise, there is no requirement that pyramid be present in all versions of the invention.

Thus, it is an object of the appended claims to cover all such modifications and variations that come within the true spirit and scope of this invention.

What is claimed is:

1. A surgical perforator, said perforator comprising:
a drive head, said drive head having features that enable a drive chuck to engage and rotate said drive head;
an inner drill that extends from said drive head for receiving a torque load that is moveably coupled to said drive head so as to have a first position wherein said drive head and said inner drill engage so that rotation of said drive head results in like rotation of said inner drill and a second position wherein rotation of said drive head does not result in rotation of said inner drill, said inner drill having:
a longitudinal center axis around which said inner drill rotates and an outer perimeter; and
at least two pairs of flutes that extend distally forward of said drive head, said flutes forming each pair of flutes being symmetrically arranged around the longitudinal center axis of said inner drill, each said flute having a first cutting surface and a first flank surface that collectively define a first cutting edge, the flank surfaces extending inwardly from the outer perimeter of said inner drill to the longitudinal center axis wherein a single said pair of flutes are formed to define a pyramid that projects forward from the first flank surfaces of said flutes from which said pyramid projects, said pyramid shaped to have cutting edges that are located forward of the cutting edges of said flutes from which said pyramid projects and the remaining said flutes being shaped to terminate outward of said pyramid so as to define a gaps between said pyramid and each remaining said flute;
an outer drill disposed around said inner drill said outer drill including: flutes defining cutting edges; at least one surface against which said inner drill, when rotated, abuts so that rotation of said inner drill results in rotation of said outer drill; and
a disengagement mechanism coupled to at least one said drive head or said inner drill for causing said inner drill to move from the first position to the second position upon the torque load applied to said inner drill falling.

2. The surgical perforator of claim 1, wherein:
said pair of flutes forming said pyramid are formed to each have a second flank surface that is angled forward from the first flank surface, the pair of second flank surfaces defining said pyramid; and
said at least one pair of flutes that do not define said pyramid are formed to each having a flank surface that terminates at a location spaced radially outwardly from said pyramid so as to define the gap between said flute and said pyramid.

3. The surgical perforator of claim 1, wherein:
each said flute has a trailing surface said such that said flute flank surfaces extend from said cutting surfaces to the trailing surfaces
said pair of flutes forming said pyramid are formed to each have: a second cutting face and a second flank surface, each second cutting face being adjacent the first cutting face of said flute and, relative to the adjacent first cutting face being angled toward the trailing surface of said flute and a second flank surface that is angled forward from the first flank surface, the pair of second faces and the pair of said second flank surfaces defining said pyramid.

4. The surgical perforator of claim 1, wherein said out drill includes at least one surface against which said inner drill moves when the torque load falls so as to cause said inner drill to move from the first position to the second position.

5. The surgical perforator of claim 1, wherein said inner drill includes two said pairs of flutes.

6. A surgical perforator, said perforator comprising:
a drive head, said drive head having features that enable a drive chuck to engage and rotate said drive head;
an inner drill for receiving a torque load that is moveably connected and extending forward from said drive head, said inner drill having: at least one leg position to engage said drive head; and at least one cutting flute located forward of said leg for forming a bore in the tissue to which said inner drill is applied, said inner drill having a first position relative to said drive head wherein said leg is engaged by said drive head so that rotation of said drive head results in like rotation of said inner drill and a second position wherein said leg is disengaged from said drive head so that rotation of said drive head does not result in rotation of said inner drill;
an outer drill for receiving a torque load that is disposed over said inner drill; and
complementary adjacent disengaging surfaces formed on said inner drill and said outer drill, said disengaging surfaces formed so that when torque load applied to said outer drill exceeds the torque applied to said inner drill, the inner drill disengaging surfaces abuts the outer drill disengaging surface so that, as the inner drill rotates, the inner drill moves from the first position to the second position and wherein said inner drill at least one leg and said disengaging surfaces are formed so that said inner drill has to rotate a maximum of 20° before said at least one leg disengages from said drive head.

7. The surgical perforator of claim 6, wherein said at least one leg is formed with a surface that functions as the disengaging surface of said inner drill.

8. The surgical perforator of claim 6, wherein said inner drill and said outer drill are formed so that, when said inner drill is rotated and the torque load applied to said inner drill is greater than the torque load applied to said outer drill, the inner drill disengaging surface abuts the outer drill torque load disengaging surface so as to rotate said outer drill.

9. The surgical perforator of claim 6, wherein a plunger is disposed in said drive head, said plunger having a stem that extends forward from said drive head, said inner drill being attached to said stem and said plunger is mounted to said drive head to be able to rotate relative to said drive head and is further able to move longitudinally within said drive head when said inner drill moves from the first position to the second position, the maximum longitudinal displacement of said plunger being 0.055 inches.

10. A surgical perforator, said perforator comprising:
a drive head, said drive head having features that enable a drive chuck to engage and rotate said drive head and a distally directed drive face, said drive face formed to define at least one notch; and
an inner drill for receiving a torque load that is moveably connected to and extends forward from said drive head, said inner drill having: at least one leg position to seat in the drive head notch; and at least one cutting flute located forward of said leg for forming a bore in the tissue to which said inner drill is applied, said inner drill having a first position relative to said drive head wherein said leg is seated in the notch and a second position wherein said leg is spaced from notch so that rotation of said drive head does not result in rotation of said inner drill;
an outer drill for receiving a torque load that is disposed over said inner drill; and
a disengagement mechanism coupled to at least one said drive head or said inner drill for causing said inner drill to move from the first position to the second position upon the torque load applied to said inner drill drops,
wherein said drive head drive face is formed so that the notch is defined by:
a distally directed base surface;
a drive wall located at one end of the base surface that extends distally away from said base surface said drive wall shaped so that when said leg is seated in the notch and said drive head is rotated in a first direction, said drive wall abuts said leg so as to rotate said leg and said inner drill; and
a ramp surface located at second end of the base surface, said ramp surface extending away from the base surface at an acute angle so that when said drive head is rotated in a second direction, said leg abuts said ramp surface.

11. The surgical perforator of claim 10, wherein:
said inner drill is formed with a plurality of said legs;
the drive head drive face is formed with a plurality of notches so that each said inner drill leg can seat in a separate notch.

12. The surgical perforator of claim 10, wherein:
said inner drill is formed with a plurality of said legs;
the drive head drive face is formed with a plurality of said notches so that each said inner drill leg can seat in a separate notch and is further formed so that between each ramp surface associated with one notch and the drive wall associated with adjacent notch there is a raised surface that extend between said notch and said drive wall that is angled relative to said ramp surface.

13. A surgical perforator, said perforator comprising:
a drive head, said drive head having features that enable a drive chuck to engage and rotate said drive head;
an inner drill that extends from said drive head for receiving a torque load that is moveably coupled to said drive head so as to have a first position wherein said drive head and said inner drill engage so that rotation of said drive head results in like rotation of said inner drill and a second position wherein rotation of said drive head does not result in rotation of said inner drill, said inner drill having a plurality of flutes, each said flute having a forwardly extending cutting face and a flank surface that angles away from the cutting face around a cutting edge, wherein the flank surface is angled away from said cutting face by at least 60°;
an outer drill disposed around said inner drill said outer drill including: flutes defining cutting edges; at least surface against which said inner drill, when rotated, abuts so that rotation of said inner drill results in rotation of said outer drill; and
a disengagement mechanism coupled to at least one said drive head or said inner drill for causing said inner drill to move from the first position to the second position upon the torque load applied to said inner drill falling

14. The surgical perforator of claim 13, wherein:
said inner drill has a longitudinal axis around which said inner drill rotates and an outer perimeter; and
said inner drill flutes are formed so as to have an inner terminus adjacent the longitudinal axis to the outer perimeter and an outer terminus adjacent the outer circumference of said inner drill, and the angle between the inner and outer terminus of said flute and the longitudinal axis of the inner drill relative to the position of the drive head is at least 70°.
